(19) United States
(12) Patent Application Publication
(10) Pub. No.: US 2025/0205312 A1
(43) Pub. Date: Jun. 26, 2025

(54) Orally Available Albenatide

(71) Applicant: AlbuNext, LLC, El Segundo, CA (US)

(72) Inventors: Patrick Soon-Shiong, Culver City, CA (US); Martin ROBITAILLE, Saint-Colomban (CA); Qinwei Wang, Culver City, CA (US); Andrew Yang, Culver City, CA (US)

(73) Assignee: AlbuNext, LLC, El Segundo, CA (US)

(21) Appl. No.: 18/982,907

(22) Filed: Dec. 16, 2024

Related U.S. Application Data
(60) Provisional application No. 63/613,625, filed on Dec. 21, 2023.

(51) Int. Cl. A61K 38/26; A61K 9/00; A61K 9/107; A61K 47/10; A61K 47/14; A61K 47/20; A61K 47/22; A61K 47/44; A61K 47/64; A61P 3/10

(52) U.S. Cl. CPC A61K 38/26; A61K 9/0053; A61K 9/107; A61K 47/10; A61K 47/14; A61K 47/20; A61K 47/22; A61K 47/44; A61K 47/643; A61P 3/10

(57) ABSTRACT

Orally administrable pharmaceutical compositions comprising an incretin or analog or derivative thereof are presented that allow for extended control of blood glucose. Formulations presented herein comprise a self-emulsifying drug delivery system (SEDDS). Advantageously such formulations afford protection from the proteolytic conditions in the upper gastrointestinal tract while allowing for effective drug release and absorption in the lower gastrointestinal tract.

This application claims priority to our copending US Provisional patent application with the Ser. No. 63/613,625, filed Dec. 21, 2023, incorporated by reference herein.

### SEQUENCE LISTING

The content of the XML file of the sequence listing named 1026900068US.xml, which is 1.67 KB in size, created on Dec. 13, 2024, and electronically submitted via EFS-Web along with the present application, is incorporated by reference in its entirety.

### FIELD OF THE INVENTION

The field of the invention is pharmaceutical compositions, especially as they relate to formulations for orally administered incretins and analogs thereof.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Regulation of blood glucose is critically important for blunting or avoiding adverse effects of temporary and/or long-term excursions of blood glucose levels in prediabetic and diabetic subjects. Beyond sulfonylurea compounds and biguanides, long and short-acting insulin is often the mainstay of treatment. Unfortunately, most forms of insulin must be injected or administered via a pump, which is generally at least inconvenient. More recently, formulations for oral insulin administration were disclosed, such as a self-emulsifying formulation (see e.g., US 2018/0311146) or a self-microemulsifying insulin delivery system (SMEDDS) (see e.g., U.S. Pat. No. 11,331,376). However, correct and responsive insulin dosing with orally administered formulations is often difficult, especially where long-term reduction of blood glucose is desired.

More recently, as an alternative to insulin treatment, various GLP-1 and GLP-1 analog-based drugs were developed and include TRULICITY™ (dulaglutide), BYETTA™ (Exenatide), VICTOZA™ (liraglutide), and OZEMPIC™ (semaglutide). Unfortunately, these drugs require injection and are often not well tolerated, especially where higher dosages are required. In contrast, RYBELSUS™ (semaglutide) is an oral preparation of semaglutide formulated with excipients such as salcaprozate sodium, povidone K90, microcrystalline cellulose, and magnesium stearate, which is orally administered once daily. However, such oral formulation typically requires fairly high quantities (14 mg) and as such is prone to side effects. More significantly, absorption of semaglutide in this formulation is easily thrown off, and the drug must therefore be taken on empty stomach at least 30 minutes before the first drink and food, and no other meds can be concurrently administered.

In still further known methods of addressing blood glucose levels, oral GLP-1 formulations using specific simple emulsion formulations are described in US 2024/0350590, where a GLP-1 hydrophobic ion pair is dissolved in an oil phase. While such formulation was shown to have strong stability in simulated gastric juice, higher stability generally of emulsions generally translates to reduced release of the GLP-1 compound and with that its bioavailability, which is especially undesirable where extended action is required. Notably, the '590 publication did not provide in vivo data establishing any efficacy.

Thus, even though various systems and methods of oral glucose management are known in the art, all or almost all of them suffer from several drawbacks. Therefore, there remains a need for improved compositions and methods for oral glucose management, especially where an incretin or analog thereof is orally administered.

### SUMMARY OF THE INVENTION

The inventors have now discovered that therapeutically effective orally administrable formulations with an incretin or analog thereof can be prepared in a simple and effective manner that allows for extended blood glucose control upon administration. In especially preferred aspects of the inventive subject matter, the incretin (e.g., GLP-1) or analog thereof (e.g., exendin-4) is formulated into a self-emulsifying drug delivery system (SEDDS).

In one aspect of the inventive subject matter, the inventors contemplate a pharmaceutical composition that comprises a hydrophobic ion pair complex comprising a hydrophobic ionic agent that is non-covalently coupled to an incretin or fragment or analog thereof. In such formulations, the hydrophobic ion pair complex is encapsulated in an emulsion of a self-emulsifying drug delivery system (SEDDS) or a SEDDS preconcentrate.

In preferred embodiments, the incretin is a Class B G-protein coupled receptor (GPCR) agonist such as a GLP-1 agonist (e.g., CJC-1134 (SEQ ID NO:1)), a GIP agonist, a GcG agonist, a NPY2 agonist, a Y2 agonist, or a PYY agonist. Most typically, the hydrophobic ionic agent is an anionic agent (e.g., docusate), and/or the SEDDS comprises n-methyl pyrrolidone, a glycol, a polyethoxylated castor oil, a mono- and/or diglyceride of a medium chain (C6-C8) fatty acid, and a non-ionic surfactant composed of a mixture of glycerides and fatty acid esters.

As will be readily appreciated, the hydrophobic ion pair complex may be encapsulated in a preconcentrate, or the SEDDS or the SEDDS preconcentrate is adsorbed onto a solid carrier. It is further preferred that the composition is prepared in a unit dosage form that provides between 100 and 1,000 mg of the SEDDS or the SEDDS preconcentrate per unit dosage. Viewed from a different perspective, the composition may also be prepared in a unit dosage form that, upon oral administration to a mammal reduces blood glucose by at least 10% over at least 6 hours.

Alternatively, in further embodiments the pharmaceutical composition may comprise an incretin or fragment or analog thereof, optionally covalently bound to a carrier protein, wherein the incretin or fragment or analog thereof is encapsulated in a pheroid drug delivery system. For example, the incretin or fragment or analog thereof is CJC-1134, which may optionally be covalently bound to a carrier protein (e.g., albumin).

In another aspect of the inventive subject matter, the inventors contemplate a method of reducing blood glucose in a mammal that includes a step of orally administering the pharmaceutical compositions as presented herein. Most typically the composition is administered (e.g., once daily) to the mammal at a dosage effective to reduce the blood glucose in the mammal.

For example, the pharmaceutical composition is formulated as a tablet or as a preconcentrate. In further embodiments, the pharmaceutical composition is formulated in a unit dose that provides between 100 and 1,000 mg of the SEDDS or the SEDDS preconcentrate per unit dosage. Thus, contemplated methods may reduce blood glucose by at least 10% over a time period of at least 6 hours, and/or reduce HbA1c by at least 0.5% (absolute) when administered over at least 8 weeks.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic illustration of an exemplary procedure for the production and experimental use of a SEDDS formulation of CJC-1134.

FIG. 2 is a graph depicting exemplary results for mean serum concentrations of various formulations of exendin-4 after IV or PO dosing.

FIG. 3 is a graph depicting exemplary results for individual blood glucose level-time profiles after single treatment.

FIG. 4 is an exemplary photomicrograph depicting CJC-1134 pre-entrapped in a pheroid formulation.

FIG. 5 is an exemplary photomicrograph depicting CJC-1134-PC-DS in a pheroid formulation.

FIG. 6 is an exemplary photomicrograph depicting CJC-1134-PC pre-entrapped in a pheroid formulation.

### DETAILED DESCRIPTION

Albenatide (CJC-1134-PC) is a peptide drug administered subcutaneously once a week chronically as a therapy for diabetes. CJC-1134, the active component of albenatide, is a modified analog of exendin-4 and acts as a potent glucagon-like peptide-1 (GLP-1) analogue. While therapeutically effective as an injectable formulation, oral formulations of CJC-1134 are nevertheless desirable as such route presents the most convenient and favorable route, and as oral formulations will reduce the cost of albenatide by avoiding recombinant human albumin and the associated conjugation process to produce the injectable albenatide formulation.

The inventors have now discovered that therapeutically effective orally administrable formulations with an incretin or analog thereof can be prepared in a simple and effective manner that allows for extended blood glucose control upon administration. In especially preferred aspects of the inventive subject matter, the incretin is a G-protein coupled receptor (GPCR) agonist, and especially a Class B GPCR agonist (e.g., GLP-1 or GLP-1 analog), that is first subjected to hydrophobic ion pairing using polar/charged side groups in the incretin or incretin analog to form a hydrophobic complex that is then formulated into a self-emulsifying drug delivery system (SEDDS) using an appropriate lipid phase.

As will be readily appreciated, especially contemplated incretins suitable for use in conjunction with the teachings presented herein will particularly include GPCR agonists (and especially Class B) and all derivatives, analogs, and fragments thereof. Therefore, and among other contemplated peptide agonists, GLP-1, GIP, Glucagon (GcG), Amylin, NPY2, Neuropeptide Y, and PYY, and derivatives, analogs, and fragments are especially preferred. It should furthermore be appreciated that the peptide agonists contemplated herein may have binding and activation specificity towards a single receptor (mono-agonist), two receptors (dual-agonist), or three receptors (tri-agonist). Consequently, it should be recognized that the biological activity of the SEDDS formulations containing such peptide agonists may be tailored towards specific uses (e.g., insulinotropic, appetite suppressant, etc.).

Therefore, and viewed from a different perspective, SEDDS formulations with GPCR (and especially Class B GPCR) agonists having desirable stability during upper gastrointestinal (GI) transit and desirable release of the agonist in the lower GI with no or little peptide loss can be prepared that activate GLP-1, GIP, GcG, NPY2, Y2, or PYY as mono-agonists, or that activate GLP-1/GIP, GLP-1/GcG, GLP-1/NPY2, GLP-1/Y2, or GLP 1/PYY as di-agonists, or that activate GLP-1/GIP/GcG as tri-agonists. Exemplary suitable agonist peptides beyond CJC-1134 (which may or may not include a linker portion and/or a coupling group) are disclosed in U.S. patent application with the Ser. No. 18/753,823, which was filed Jun. 25, 2024, and which is incorporated in its entirety by reference here. Furthermore, due to the lipophilic nature of the SEDDS formulation it is contemplated that the agonist peptides can be efficiently delivered to various tissues, including neural tissue, hepatic tissue, adipose tissue, and/or pancreatic tissue.

In at least some embodiments, it is preferred that the peptide agonist will be exendin-4 or an exendin-4 derivative that includes a modified amino acid to avoid peptide cleavage by dipeptidyl peptidase-4, and that includes a C-terminal tryptophan cage (‘fish-hook’) structure to further enhance stability. Moreover, and depending on the particular amino acid substitutions (relative to exendin-4), contemplated peptide agonists will exhibit equal or higher affinity than native GLP-1 to the GLP-1 receptor, resulting in higher potency. Additionally, one or more amino acid substitutions (relative to exendin-4) may further enable multi-agonist activity (e.g., tri-agonist activity towards GLP-1/GIP/Glucagon receptors). In still further examples, the N- and/or C-terminus of the peptide may be modified with one or more of lysine, histidine, or arginine to introduce a positive charge, or one or more of glutamic acid or aspartic acid to introduce a negative charge. These charges can advantageously be used to serve as anchor points to hydrophobic ion pairing.

Alternatively or additionally, the C-terminal lysine (or other N- and/or C-terminal amino acid) may optionally be further modified to include a linker group that may or may not contain a chemical group with one or more positive or negative charges to further enhance hydrophobic ion pairing. In one example, an optionally modified lysine may form a peptide bond with the C-terminal serine of exendin-4 or other peptide where the modified lysine further includes a linker that may or may not be covalently bound to a chemical group containing one or more positive or negative charges (e.g., containing one or more of lysine, arginine, and histidine, or containing one or more of glutamic acid or aspartic acid), or a chemically reactive group such as MPA (maleimidopropionic acid). Of course, it should be appreciated that various alternative linker moieties may be used, and suitable alternative linkers with optional coupling groups (such as MPA, which may be omitted or replaced by another group in the examples below) include AEEA, AEEA-MPA, AEEA-(bromo) MPA, (AEEA) 2-MPA, (AEEA) 2-(bromo) MPA, AEEA-OA-MPA, AEEA-OA-(bromo) MPA, AEEA-OA-AEEA-MPA, AEEA-OA-AEEA-(bromo) MPA, (AEEA)₂-OA-MPA, and (AEEA)₂-OA-(bromo) MPA, with AEEA denoting 2-[2-(2-aminoethoxy) ethoxy]acetic acid, and with OA denoting 8-aminooctanoate.

Where the agonist peptide (such as CJC-1134) includes a linker and a coupling group, it is particularly contemplated that such agonist peptide may be covalently coupled vial the coupling group to albumin, and especially human albumin (resulting, for example, in CJC-1134-PC, albenatide). Such covalent coupling may be conventional, or in a retro-Michael resistant manner. Exemplary suitable albumin conjugates are described in U.S. patent application Ser. No. 18/753,823, filed Jun. 25, 2024 and Ser. No. 18/753,928, filed Jun. 25, 2024, both incorporated in their entirety by reference herein.

Depending on the number of charged amino acids and their charge, it should be appreciated that the hydrophobic ion pairing can be performed with a variety of hydrophobic anionic and cationic reagents in a manner such that a positive charge at an amino acid in the peptide will bind to an anionic group of the hydrophobic reagent, and such that a negative charge at an amino acid in the peptide will bind to an cationic group of the hydrophobic reagent. Moreover, it is generally contemplated that each of the peptides presented herein may have multiple ion pairings, for example, two, or three, or four, or five, and even more.

Among other suitable hydrophobic anionic reagents, particularly contemplated anionic reagents include docusate (dioctyl sulfosuccinate, DOC), sodium octyl sulfate (SOS), 1-hydroxy-2-naphthoic acid (xinafoic acid), 2-naphthalene sulfonic acid (NSA), brilliant blue FCF, carboxy methyl polyethylene glycol (CM-PEG), cholesteryl hemisuccinate, cholic acid, sodium cholate, decanoic acid, sodium decanoate, sodium caprate, dimyristoyl phosphatidylglycerol (DMPG), dioleoyl phosphatidic acid (DOPA), docosahexaenoic acid, hexadecyl phosphate, linoleic acid, N, N-dipalmitoyl-L-lysine, oleic acid, sodium oleate, pamoic acid, disodium pamoate, sodium acetate, sodium cholesteryl sulfate, sodium decanesulfonate (SDES), sodium deoxycholate, sodium docusate, sodium dodecyl benzenesulfonate (SDBS), sodium dodecyl sulfate, sodium laurate, sodium n-octadecyl sulfate, sodium stearate, sodium stearoyl glutamate (SSG), sodium taurodeoxycholate (STDC), sodium tetradecyl sulfate, sodium tripolyphosphate, taurocholic acid, sodium taurocholate, and/or vitamin E succinate. Additional non-limiting examples of hydrophobic anionic reagents include diacylphosphatidylglycerol derivatives such as dimyristoyl-, dioleyl-, dipalmitoyl- and distearoyl phosphatidylglycerols, -tocopheryl succinate, tocopheryl phosphate, sodium dioctylsulfosuccinate, mono- and disubstituted cetylphosphates, cholates, deoxycholates, ammonium glycyrrhizinate, cholesteryl hemisuccinate, cholesteryl sulfate, and cholesteryl sulfate.

Similarly, numerous hydrophobic cationic reagents are contemplated, and exemplary cationic reagents include cations of arginine-hexadecanoyl ester (AHE), arginine-nonyl ester (ANE), N-benzyl-2-phenylethanamine, chitosan, dodecylamine, hexadecyl trimethylammonium bromide (CTAB), maprotiline, Na-deoxycholyl-L-lysyl-methylester, N, N′-dibenzyl ethylenediamine, N, N-dimethyl dodecylamine, N, N-dimethyl hexylamine, N, N-dimethyl octadecylamine, stearylamine, tetrabutyl e (TBAB), tetraheptyl ammonium bromide (THA), tetrahexyl ammonium bromide, tetraoctyl ammonium bromide (TOAB), tetrapentyl ammonium bromide (TPA), and/or triethylamine (TEA).

With respect to suitable lipid components for the SEDDS formulations presented herein it is contemplated that preferred components include medium (C8-C12) and long chain (C14-C18) triglycerides, various mono- and di-glycerides, pegylated esters, and ethoxylated oils. Viewed from a different perspective, contemplated SEDDS compositions may be categorized into one of four classes: (1) Formulations with 100% of lipids. (2) Formulations without water-soluble components comprising 40-80% oils and 20-60% surfactants with low hydrophilic-lipophilic balance (HLB) values. Such formulations are advantageously easily digested. (3) Formulations with water-soluble components, typically comprising <20-80% oils, 20-50% high-HLB surfactants, and 0-50% hydro cosolvents. (4) Formulations comprising 0-20% low-HLB surfactants, 30-80% high-HLB surfactants, and 0-50% hydro cosolvents. Most typically, but not necessarily, contemplated formulations will include various lipids as solubilizers, emulsifiers, surfactants, and potentially co-surfactants, various medium-chain triglycerides acting as solubilizers, various mono- and di-glycerides acting as solubilizers and emulsifiers, and/or various pegylated esters, polysorbates, and ethoxylated oils as surfactants and co-surfactants.

Therefore, and among various other options, suitable SEDDS ingredients will include propylene glycol, N-methyl pyrrolidone, Labrasol (caprylocaproyl polyoxyl-8 glycerides), Gelucire 44/14 (lauroyl polyoxyl-32 glycerides), Labrafil series (oleoyl, linoleoyl, or linolenoyl polyoxyl-6 glycerides NF), Cremophor EL, Capmul MCM, Transcutol (diethylene glycol monoethyl ether), and castor oil.

For preparation of the hydrophobic ion pairing, it is generally contemplated that the hydrophobic ionic agent will be present in molar excess relative to the peptide, and suitable ratios include 2:1-3:1, or 3:1-4:1, or 4:1-5:1, or 5:1-6:1, or 6:1-7-1, and in some cases even higher. However, and depending on the particular peptide chosen, the ration may also be below 2:1, and even below 1:1. Most typically, but not necessarily, the so prepared ion-pair is then lyophilized or otherwise dehydrated prior to admixture with the lipid component, especially where the SEDDS system is prepared without water soluble components. Depending on the particular ion-pair and lipids chosen, the ion-pair is then dissolved in the preconcentrate at a concentration of at least 0.1%, or at least 0.3%, or at least 0.6%, or at least 0.9%, or at least 1.2%, or at least 1.5%, or at least 1.8%, or at least 2.1%, and in some cases even higher. As will be readily appreciated, the preconcentrate can then be added to an aqueous medium to then form the SEDDS as is well known in the art.

In still further contemplated aspects of the inventive subject matter it should be noted that the SEDDS formulations may include one or more absorption enhancers to increase transfer of the peptides from the lower GI tract into systemic circulation and organ systems. Among other absorption enhancers, especially contemplated absorption enhancers include ethylenediamine tetraacetic acid (EDTA), citric acid, salicylate, sodium dodecyl sulfate (SDS), sodium deoxycholate, sodium taurocholate, oleic acid, acylcarnitine, sodium caprate, sodium caprylate, salcaprozate (SNAC), 5-CNAC, dimyristoyl phosphatidylglycerol (DMPG), cetyltrimethylammonium bromide (CTAB) and/or phospholipids. Additionally, and where desired or needed it is contemplated that the SEDDS formulations may also include one or more antioxidants, and particularly preferred antioxidants include lipophilic antioxidants such as acetyl methionine, quercetin, alpha-tocopherol, various carotenoids, ubiquinol, docosahexaenoic acid, and eicosapentaenoic acid.

To stabilize the SEDDS formulation, it is also contemplated that one or more encapsulants may be included to protect the SEDDS formulation from rapid degradation. Most typically, suitable encapsulants include various polymers and particularly polysaccharides such as pectins, alginic acid, dextrans, and cyclic polysaccharides such as cyclodextrins (especially hydroxypropyl-beta-cyclodextrin). Alternatively, or additionally, suitable encapsulants also include protein-based polymers, and particularly gelatin and collagens.

In further embodiments of the inventive subject matter, the inventors contemplate encapsulation of the therapeutic peptides and protein conjugates in a pheroid formulation to so allow for oral administration of the therapeutic peptide and release and absorption of the peptide from the GI tract upon oral administration (see e.g., Toxicology Reports 6 (2019) 940-950; incorporated by reference herein). Most typically, for pre-encapsulated (self-emulsifying) systems and for pheroid emulsions, fatty acids will typically be used as vitamin E ethyl ester, along with PEG 400, Incromega E3322 (EPA/DHA concentrate containing a minimum of 65% omega 3) and E7010 (70 percent concentrate of eicospentaenoic acid). Further ingredients may include dl-Alpha tocopherol, Kolliphor EL (Macrogolglycerol ricinoleate), and preservatives (e.g., methylparaben and propylparaben) and/or antioxidants (butylatedhydroxyanisole and butylatedhydroxytoluene) as desired or needed. All formulations are typically gassed with nitrous oxide gas.

With respect to suitable formulations, it is contemplated that the SEDDS and pheroid compositions presented herein are typically formulated for oral administration, and most preferably in solid or liquid form. For example, contemplated SEDDS compositions may be filled into capsules or may be adsorbed onto a pharmaceutically acceptable solid carrier to so form a solid drug formulation. Among other solid carriers, (porous) microcrystalline cellulose, (nanosized) amorphous silicon dioxide, (oolithic) aragonite having an average size of between 5 and 500 micrometers, and magnesium aluminometasilicate are deemed suitable for use herein.

Most typically, the SEDDS and pheroid compositions are formulated into unit doses in a solid form that each contain between 1-10 mg, or between 10-100 mg, or between 100-500 mg, or between 500-1,000 mg of the peptide. Thus, and viewed from a different perspective, a unit dose may contain at least 1 mg or at least 10 mg, or at least 50 mg, or at least 100 mg, or at least 500 mg, or at least 1,000 mg (typically corresponding to between 0.1-1.0 mg/kg, or between 1.0 and 5.0 mg/kg, or between 5.0-10 mg/kg, and even higher).

Notably, such prepared SEDDS compositions have shown extended activity to reduce blood glucose in vivo, and even to reduce HbA1c as shown in more detail below. For example, and depending on the dosage and type of peptide used, it is contemplated that fasting blood glucose can be reduced by at least 5%, or by at least 10%, or by at least 15%, or by at least 20%, or by at least 25% over control (no drug administered) over a period of at least 4 hours, at least 6 hours, or at least 8 hours, or at least 12 hours, or at least 18 hours, or at least 24 hours. Likewise, it is contemplated that over extended administration of contemplated SEDDS formulations HbA1c can be reduced by at least 0.5%, or at least 0.7%, or at least 0.9%, or at least 1.2%, or at least 1.5% (absolute) where the extended administration spans over at least 4 weeks, or at least 8 weeks, or at least 12 weeks. Most preferably, administration will be once daily during such administration.

### Examples

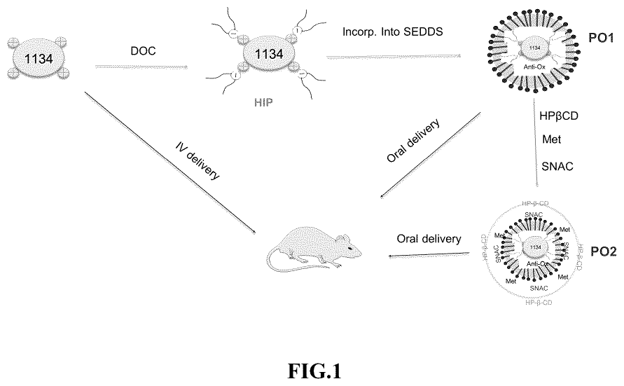

The following examples provide exemplary guidance to illustrate various aspects of the inventive subject matter and are not intended to limit the scope of this disclosure. As is shown in more detail below, a sufficient degree of lipophilicity of a peptide drug is important for the successful incorporation into an oily droplet core in an SEDDS. Accordingly, all or almost all of the embodiments contemplated herein will include hydrophobic ion pairing in which ionic interactions between the incretin peptide (or analog thereof) and an oppositely charged surfactant was utilized. FIG. 1 schematically depicts an exemplary procedure for the production and experimental use of a SEDDS formulation of CJC-1134.

In the following examples, and unless indicated otherwise, a lipid-based drug delivery system SEDDS was developed for CJC-1134, a synthetic peptide having the sequence HGEGTFTSDLSKQMEEEAVRLFIEWLKNGGPSSGAPPPSK (SEQ ID NO:1). The peptide was successfully ion-paired with the commercially available counterion docusate (DOC), before being incorporated into the SEDDS.

Hydrophobic ion pairing of CJC-1134: Hydrophobic ion pairing based on ionic interactions between the peptide and oppositely charged surfactants. One advantage is that it does not involve chemical modification of the peptide. The strategy of hydrophobic ion pairing was applied to increase lipophilicity of CJC-1134 and facilitate incorporation into SEDDS. The universal counterion of sodium docusate (DOC) was used and the CJC-1134/DOC ion pair was successful obtained. The peptide contents and purity were analyzed by HPLC.

Preparation of SEDDS: For SEDDS generation, the ion-pair CJC-1134/DOC in a ratio of 1:5 was chosen. A previously developed SEDDS formulation exhibiting protection against enzymatic degradation (data not shown) was employed. The proportions of the individual components were slightly adjusted in order to identify the most suitable composition for the incorporation of the ion pair. The lyophilized CJC-1134/DOC was dissolved in a concentration of 1.5% in the pre-concentrate for in vitro studies. On the other hand, to protect the peptide from oxidation, 1.5% acetyl methionine was incorporated into the SEDDS formulation as well. For in vivo studies, the ion-pair CJC-1134/DOC in SEDDS pre-concentrates were emulsified in a ratio of 1:7.5 with DI water before application in order to reach the applicable peptide concentration of 1.0 mg/ml.

To evaluate if the permeation enhancer SNAC can further increase the oral bioavailability, in a separate formulation, SNAC with 15-fold of ion pair and a solid carrier HPβCD with 100-fold of ion pair were added. For comparison, an exendin-4/DOC ion pair was prepared and incorporated in the same SEDDS formulation as described above. Table 1 shows the SEDDS components of the formulations with and without incorporated ion pairs.

TABLE 1

| Formulation | components | Amount (%) |
|---|---|---|
| SEDDS vehicle | NMP | 3 |
| | Propylene glycol | 7 |
| | Cremophor EL | 37 |
| | Capmul MCM | 26.5 |
| | Labrafil 1944 | 26.5 |
| CJC-1134-PO1 | SEDDS vehicle | 97 |
| | CJC-1134/DOC | 1.5 |
| | AcMet | 1.5 |
| CJC-1134-PO2 | SEDDS vehicle | 97 |
| | CJC-1134/DOC | 1.5 |
| | AcMet | 1.5 |
| | Additional components | SNAC (15xExe/DOC); |
| | | HPβCD (100xExe/DOC) |
| Exendin-4-PO1 | SEDDS vehicle | 97 |
| | Exendin-4/DOC | 1.5 |
| | AcMet | 1.5 |
| Exendin-4-PO2 | SEDDS vehicle | 97 |
| | Exendin-4/DOC | 1.5 |
| | AcMet | 1.5 |
| | Additional components | SNAC (15xExe/DOC); |
| | | HPβCD (100xExe/DOC) |

### Pilot In Vivo Studies:

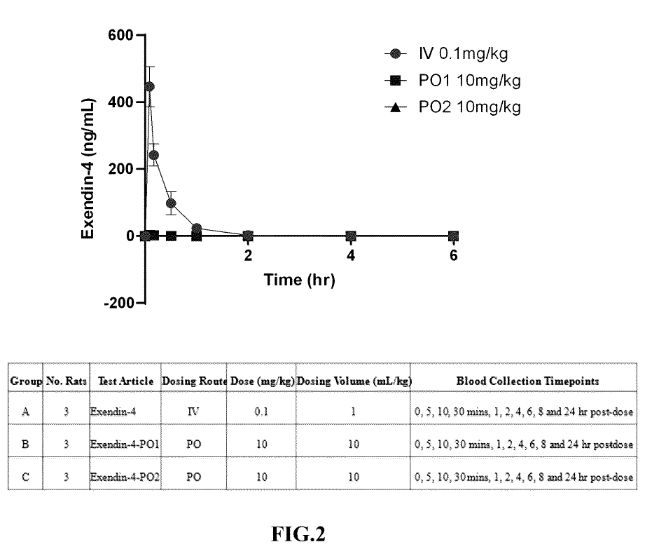

For pharmacokinetic studies, rats were randomly divided into 3 groups (n=3). The first group received a dose of 0.1 mg/kg bodyweight of exendin-4 solution in 0.9% saline via s.c. injection. SEDDS formulations, Exendin-4-PO1 and Exeendin-4-PO2, containing the HIP in a dose of 10 mg/kg bodyweight were administered via oral gavage to the second and third group, respectively. SEDDS preconcentrates were emulsified in a ratio of 1:7.5 with water before application. The study protocol is described in Table 2. The Mean serum concentration-time profiles of Exendin-4 after IV or PO dose are listed in Table 3, Table 4, and Table 5, and are also shown in FIG. 2.

TABLE 2

| | No. | Test | Dosing | Dose | Dosing Volume | |
|---|---|---|---|---|---|---|
| Group | Rats | Article | Route | (mg/kg) | (mL/kg) | Blood Collection Timepoints |
| A | 3 | Exendin-4 | IV | 0.1 | 1 | 0, 5, 10, 30 mins, 1, 2, 4, 6, 8 and 24 hr |
| | | | | | | post-dose |
| B | 3 | Exendin- | PO | 10 | 10 | 0, 5, 10, 30 mins, 1, 2, 4, 6, 8 and 24 hr |
| | | 4-PO1 | | | | post-dose |
| C | 3 | Exendin- | PO | 10 | 10 | 0, 5, 10, 30 mins, 1, 2, 4, 6, 8 and 24 hr |
| | | 4-PO2 | | | | post-dose |

TABLE 3
Individual and mean serum concentration-time data of Exendin-4 after
an IV dose of Exendin-4 at 0.1 mg/kg to male SD rats (N = 3)

| dose level | Sampling | concentration(ng/mL) | | | Mean | | |
|---|---|---|---|---|---|---|---|
| (mg/kg) | Time (hr) | Rat#101 | Rat#102 | Rat#103 | (ng/mL) | SD | CV (%) |
| 0.1 | predose | BLQ | BLQ | BLQ | BLQ | NA | NA |
| | 0.0833 | 476 | 378 | 488 | 447 | 60.4 | 13.5 |
| | 0.167 | 218 | 228 | 281 | 243 | 33.9 | 14.0 |
| | 0.5 | 84.9 | 71.7 | 137 | 98.0 | 34.8 | 35.5 |
| | 1 | 21.2 | 17.8 | 31.8 | 23.6 | 7.34 | 31.1 |
| | 2 | 2.21 | 1.65 | 3.24 | 2.37 | 0.809 | 34.2 |
| | 4 | 0.218 | 0.208 | BLQ | 0.213 | NA | NA |
| | 6 | BLQ | BLQ | BLQ | BLQ | NA | NA |
| | 8 | BLQ | BLQ | BLQ | BLQ | NA | NA |
| | 24 | BLQ | BLQ | BLQ | BLQ | NA | NA |
| PK parameters | Unit | Rat#101 | Rat#102 | Rat#103 | Mean | SD | CV (%) |
| Rsq_{_{—}}_{adjusted} | | 0.947 | 0.924 | 0.996 | 0.956 | 0.0367 | 3.84 |
| CL | mL/hr/kg | 546 | 662 | 457 | 555 | 103 | 18.5 |
| Vₛₛ | mL/kg | 156 | 197 | 151 | 168 | 25.5 | 15.2 |
| T_{1/2} | hr | 0.389 | 0.365 | 0.282 | 0.345 | 0.0564 | 16.3 |
| AUC₍₀₋ₜ₎ | hr*ng/mL | 183 | 151 | 217 | 184 | 33.2 | 18.1 |
| AUC_{(0-∞)} | hr*ng/mL | 183 | 151 | 219 | 184 | 33.8 | 18.3 |
| MRT₍₀₋ₜ₎ | hr | 0.284 | 0.295 | 0.317 | 0.299 | 0.0170 | 5.68 |
| MRT_{(0-∞)} | hr | 0.287 | 0.298 | 0.330 | 0.305 | 0.0223 | 7.31 |

TABLE 4
Individual and mean serum concentration-time data of Exendin-4 after
a PO dose of Exendin-4-PO1 at 10 mg/kg to male SD rats (N = 3)

| dose level | Sampling | concentration(ng/mL) | | | Mean | | |
|---|---|---|---|---|---|---|---|
| (mg/kg) | Time (hr) | Rat#201 | Rat#202 | Rat#203 | (ng/mL) | SD | CV (%) |
| 10 | predose | BLQ | BLQ | BLQ | BLQ | NA | NA |
| | 0.0833 | 9.09 | 0.291 | 0.408 | 3.26 | 5.05 | 155 |
| | 0.167 | 5.12 | BLQ | 0.211 | 2.67 | NA | NA |
| | 0.5 | 1.03 | 0.217 | 0.213 | 0.485 | 0.468 | 96.4 |
| | 1 | 0.300 | BLQ | BLQ | 0.300 | NA | NA |
| | 2 | 0.202 | 0.263 | BLQ | 0.232 | NA | NA |
| | 4 | BLQ | 0.239 | BLQ | 0.239 | NA | NA |
| | 6 | BLQ | BLQ | BLQ | BLQ | NA | NA |
| | 8 | 2.22* | BLQ | BLQ | BLQ | NA | NA |
| | 24 | BLQ | BLQ | BLQ | BLQ | NA | NA |
| PK parameters | Unit | Rat#201 | Rat#202 | Rat#203 | Mean | SD | CV (%) |
| Rsq_{_{—}}_{adjusted} | | 0.660 | NA | NA | 0.660 | NA | NA |
| Tₘₐₓ | hr | 0.0833 | 0.0833 | 0.0833 | 0.0833 | 0.00 | 0.00 |
| Cₘₐₓ | ng/mL | 9.09 | 0.291 | 0.408 | 3.26 | 5.05 | 155 |
| T_{1/2} | hr | 0.433 | NA | NA | 0.433 | NA | NA |
| AUC₍₀₋ₜ₎ | hr*ng/mL | 2.58 | 0.979 | 0.114 | 1.22 | 1.25 | 102 |
| AUC_{(0-∞)} | hr*ng/mL | 2.70 | NA | NA | 2.70 | NA | NA |
| MRT₍₀₋ₜ₎ | hr | 0.342 | 2.03 | 0.246 | 0.872 | 1.00 | 115 |
| MRT_{(0-∞)} | hr | 0.449 | NA | NA | 0.449 | NA | NA |
| F | % | 0.0140 | 0.00533 | 0.000618 | 0.00666 | 0.00680 | 102 |

TABLE 5
Individual and mean serum concentration-time data of Exendin-4 after
a PO dose of Exendin-4-PO2 at 10 mg/kg to male SD rats (N = 3)

| dose level | Sampling | concentration(ng/mL) | | | Mean | | |
|---|---|---|---|---|---|---|---|
| (mg/kg) | Time (hr) | Rat#301 | Rat#302 | Rat#303 | (ng/mL) | SD | CV (%) |
| 10 | predose | BLQ | BLQ | BLQ | BLQ | NA | NA |
| | 0.0833 | 4.18 | 1.16 | 3.70 | 3.01 | 1.62 | 53.8 |
| | 0.167 | 4.49 | 0.559 | 5.18 | 3.41 | 2.49 | 73.1 |
| | 0.5 | 0.976 | BLQ | 0.629 | 0.802 | NA | NA |
| | 1 | 0.319 | BLQ | 0.222 | 0.270 | NA | NA |
| | 2 | BLQ | BLQ | 0.215 | 0.215 | NA | NA |
| | 4 | BLQ | BLQ | 0.208 | 0.208 | NA | NA |
| | 6 | BLQ | BLQ | BLQ | BLQ | NA | NA |
| | 8 | BLQ | BLQ | BLQ | BLQ | NA | NA |
| | 24 | BLQ | BLQ | BLQ | BLQ | NA | NA |
| PK parameters | Unit | Rat#301 | Rat#302 | Rat#303 | Mean | SD | CV (%) |
| Rsq_{_{—}}_{adjusted} | | 1.00 | NA | 0.929 | 0.964 | NA | NA |
| Tₘₐₓ | hr | 0.167 | 0.0833 | 0.167 | 0.139 | 0.0481 | 34.6 |
| Cₘₐₓ | ng/mL | 4.49 | 1.16 | 5.18 | 3.6 | 2.15 | 59.5 |
| T_{1/2} | hr | 0.310 | NA | 34.6 | 17.4 | NA | NA |
| AUC₍₀₋ₜ₎ | hr*ng/mL | 1.77 | 0.120 | 2.34 | 1.41 | 1.15 | 81.8 |
| AUC_{(0-∞)} | hr*ng/mL | 1.91 | NA | 12.7 | 7.32 | NA | NA |
| MRT₍₀₋ₜ₎ | hr | 0.264 | 0.0995 | 0.844 | 0.403 | 0.391 | 97.1 |
| MRT_{(0-∞)} | hr | 0.352 | NA | 44.1 | 22.2 | NA | NA |
| F | % | 0.00963 | 0.000653 | 0.0128 | 0.00768 | 0.00628 | 81.8 |

For pharmacodynamics studies, db/db mice were randomly divided into 5 groups (n=3). The test article preparation and dosing procedure followed the same way as described in PK study above. The study protocol is described in Table 6. Individual blood glucose level-time profiles after CJC-1134 and Exendin-4 dose are listed in Table 7 and shown in FIG. 3.

TABLE 6

| | | | Dosing | Dose | Dosing Vol | |
|---|---|---|---|---|---|---|
| Group | Mice# | Test Article | Route | (mg/kg) | (mL/kg) | Blood Glucose Test Timepoints |
| A | 3 | Vehicle | SC | 0 | 8 | 0, 15, 30 mins, 1, 3, 6, 8 and 24 hr post- |
| | | | | | | dose |
| B | 3 | Exendin-4-PO-1 | PO | 10 | 10 | 0, 15, 30 mins, 1, 3, 6, 8 and 24 hr post- |
| | | | | | | dose |
| C | 3 | CJC-1134-DAC | SC | 0.8 | 8 | 0, 15, 30 mins, 1, 3, 6, 8 and 24 hr post- |
| | | | | | | dose |
| D | 3 | CJC-1134-DAC- | PO | 10 | 10 | 0, 15, 30 mins, 1, 3, 6, 8 and 24 hr post- |
| | | PO1 | | | | dose |
| E | 3 | CJC-1134-DAC- | PO | 10 | 10 | 0, 15, 30 mins, 1, 3, 6, 8 and 24 hr post- |
| | | PO2 | | | | dose |

TABLE 7

| | | Blood Glucose Level (mmol/L) | | | | | | | | |
|---|---|---|---|---|---|---|---|---|---|---|
| | | 0 h | | | | | | | | |
| | | after | | | | | | | | AUC |
| Group | NO. | fasting | 0.25 h | 0.5 h | 1 h | 3 h | 6 h | 8 h | 24 h | mmol/L · h |
| G1: Vehicle- | 1-1 | 21.9 | 27.5 | 29.3 | 26.7 | 32.9 | 26.1 | 29.4 | 29.9 | 705.3 |
| SC | 1-2 | 15.0 | 25.5 | 28.6 | 31.1 | 33.9 | 27.5 | 31.7 | 28.9 | 727.9 |
| | 1-3 | 20.3 | 29.2 | 33.1 | 34.5 | 35.5 | 34.1 | 30.3 | 32.7 | 773.7 |
| | Mean | 19.1 | 27.4 | 30.3 | 30.8 | 34.1 | 29.2 | 30.5 | 30.5 | 735.6 |
| | SD | 3.61 | 1.85 | 2.42 | 3.91 | 1.31 | 4.27 | 1.16 | 1.97 | 34.85 |
| G2: Exendin- | 2-1 | 7.4 | 12.9 | 9.4 | 7.0 | 13.6 | 10.9 | 16.7 | 25.6 | 432.8 |
| 4-PO1 | 2-2 | 23.3 | 29.5 | 26.5 | 20.8 | 29.6 | 31.1 | 29.6 | 34.5 | 740.4 |
| (10 mg/kg) | 2-3 | 23.0 | 28.9 | 26.2 | 22.6 | 32.2 | 31.7 | 31.7 | 17.3 | 631.6 |
| | Mean | 17.9 | 23.8 | 20.7 | 16.8 | 25.1 | 24.6 | 26.0 | 25.8 | 601.6 |
| | SD | 9.09 | 9.42 | 9.79 | 8.53 | 10.07 | 11.84 | 8.12 | 8.60 | 155.98 |
| | T- | 0.8465 | 0.5478 | 0.1733 | 0.0615 | 0.2010 | 0.5557 | 0.3991 | 0.4085 | 0.2201 |
| | TESTvsG1 | | | | | | | | | |
| G3: CJC- | 3-1 | 8.7 | 20.5 | 18.8 | 11.5 | 10.4 | 12.7 | 11.3 | 17.0 | 323.1 |
| 1134-SC | 3-2 | 16.4 | 21.2 | 21.0 | 16.5 | 14.0 | 18.7 | 16.3 | 16.9 | 399.5 |
| (0.8 mg/kg) | 3-3 | 20.6 | 25.6 | 19.0 | 15.9 | 16.1 | 17.5 | 11.4 | 18.4 | 369.8 |
| | Mean | 15.2 | 22.4 | 19.6 | 14.6 | 13.5 | 16.3 | 13.0 | 17.4 | 364.1 |
| | SD | 6.04 | 2.76 | 1.22 | 2.73 | 2.88 | 3.17 | 2.86 | 0.84 | 38.52 |
| | T- | 0.3986 | 0.0610 | 0.0024 | 0.0042 | 0.0004 | 0.0136 | 0.0006 | 0.0005 | 0.0002 |
| | TESTvsG1 | | | | | | | | | |
| G4: CJC- | 4-1 | 21.8 | 29.3 | 28.5 | 26.2 | 29.0 | 25.9 | 24.0 | 31.4 | 657.9 |
| 1134-PO1 | 4-2 | 10.1 | 23.2 | 17.1 | 11.1 | 24.6 | 21.9 | 21.8 | 32.9 | 603.0 |
| (10 mg/kg) | 4-3 | 22.6 | 32.5 | 31.8 | 33.4 | 34.2 | 33.5 | 30.1 | 33.1 | 769.6 |
| | Mean | 18.2 | 28.3 | 25.8 | 23.6 | 29.3 | 27.1 | 25.3 | 32.5 | 676.8 |
| | SD | 7.00 | 4.72 | 7.71 | 11.38 | 4.81 | 5.89 | 4.30 | 0.93 | 84.88 |
| | T- | 0.8527 | 0.7660 | 0.3864 | 0.3586 | 0.1681 | 0.6384 | 0.1149 | 0.1929 | 0.3295 |
| | TESTvsG1 | | | | | | | | | |
| G5: CJC- | 5-1 | 15.1 | 26.1 | 23.6 | 22.0 | 26.0 | 28.1 | 21.6 | 32.7 | 636.0 |
| 1134-PO2 | 5-2 | 25.7 | 31.5 | 27.7 | 25.5 | 31.9 | 28.1 | 27.9 | 34.0 | 726.5 |
| (10 mg/kg) | 5-3 | 22.6 | 32.5 | 29.2 | 31.2 | 32.4 | 28.6 | 25.8 | 30.5 | 689.6 |
| | Mean | 21.1 | 30.0 | 26.8 | 26.2 | 30.1 | 28.3 | 25.1 | 32.4 | 684.0 |
| | SD | 5.45 | 3.44 | 2.90 | 4.64 | 3.56 | 0.29 | 3.21 | 1.77 | 45.48 |
| | T- | 0.6132 | 0.3081 | 0.1838 | 0.2655 | 0.1418 | 0.7157 | 0.0527 | 0.2818 | 0.1939 |
| | TESTvsG1 | | | | | | | | | |

For the pharmacokinetic (PK) study, oral administration of Exendin-4/DOC formulated in SEDDS to Sprague Dawley (SD) rats, no apparent absorption was observed as compared with a free exendin-4 solution. In the pharmacodynamic (PD) study, the SEDDS of CJC-1134 (PO1) caused more than 20% lower blood glucose level one hour after dosing, compared to the control only. After 8 hours, blood glucose levels in the CJC-1134/DOC (PO1) SEDDS group were still lower as compared to the control. Notably, no additional effect was observed by including salcaprozate (SNAC) in the test article. According to these results, there is evidence that incorporation in SEDDS could enhance oral bioavailability of CJC-1134 as demonstrated in the PD study.

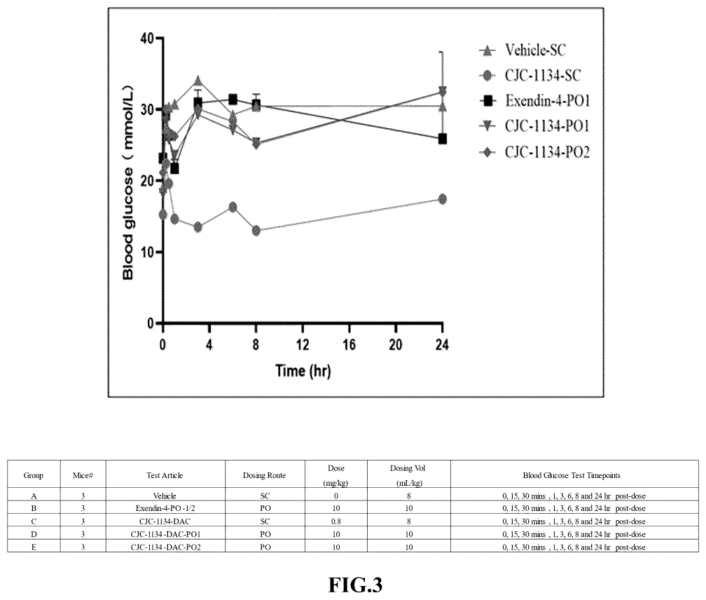

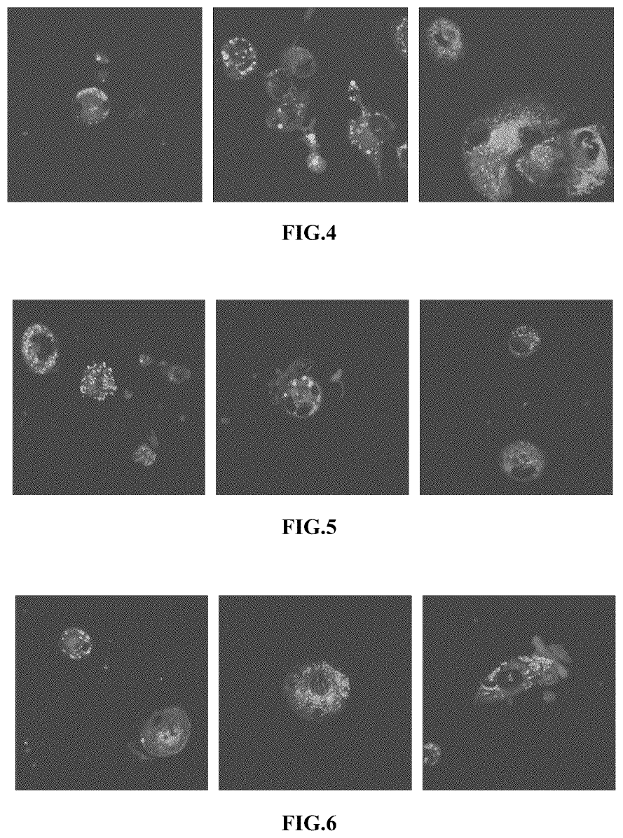

In an alternate approach, CJC-1134 and the corresponding albumin conjugate CJC-1134-PC were formulated into orally administrable formats using pheroid technology (see e.g., Toxicology Reports 6 (2019) 940-950), and exemplary results are shown in FIG. 4-6. More particularly, FIG. 4 is a photomicrograph showing unconjugated CJC-1134 peptide pre-entrapped in pheroid visualized with Nile Red, while FIG. 5 is a photomicrograph showing the corresponding CJC-1134-PC-DS albumin conjugate in a pheroid formulation visualized with Nile Red. FIG. 6 is a photomicrograph showing the CJC-1134-PC albumin conjugate pre-entrapped in pheroid visualized with Nile Red.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, suitable calculations of the percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100. Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridizes to filter-bound DNA or RNA in 3× sodium chloride/sodium citrate (SSC) at approximately 45° C. followed by at least one wash in 0.2×SSC/0.1% SDS at approximately 20-65° C.

Alternatively, a substantially similar polypeptide may differ by at least 1, but less than 5, 10, 20, 50 or 100 amino acids from the sequences shown in, for example, in the amino acid sequence that are included within SEQ ID No: 1. Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A pharmaceutical composition, comprising:
a hydrophobic ion pair complex comprising a hydrophobic ionic agent that is non-covalently coupled to an incretin or fragment or analog thereof;
wherein the hydrophobic ion pair complex is encapsulated in an emulsion of a self-emulsifying drug delivery system (SEDDS) or a SEDDS preconcentrate.

2. The composition of claim 1, wherein the incretin is a Class B G-protein coupled receptor (GPCR) agonist.

3. The composition of claim 1, wherein the incretin is a GLP-1 agonist, a GIP agonist, a GcG agonist, a NPY2 agonist, a Y2 agonist, or a PYY agonist.

4. The composition of claim 1, wherein the incretin is CJC-1134 (SEQ ID NO:1).

5. The composition of claim 1, wherein the hydrophobic ionic agent is an anionic agent.

6. The composition of claim 5, wherein the hydrophobic ionic agent is docusate.

7. The composition of claim 1, wherein the SEDDS comprises n-methyl pyrrolidone, a glycol, a polyethoxylated castor oil, a mono- and/or diglyceride of a medium chain (C6-C8) fatty acid, and a non-ionic surfactant composed of a mixture of glycerides and fatty acid esters.

8. The composition of claim 1, wherein the hydrophobic ion pair complex is encapsulated in a preconcentrate.

9. The composition of claim 1, wherein the SEDDS or the SEDDS preconcentrate is adsorbed onto a solid carrier.

10. The composition of claim 9, wherein the composition is prepared in a unit dosage form that provides between 100 and 1,000 mg of the SEDDS or the SEDDS preconcentrate per unit dosage.

11. The composition of claim 9, wherein the composition is prepared in a unit dosage form that, upon oral administration to a mammal reduces blood glucose by at least 10% over at least 6 hours.

12. A method of reducing blood glucose in a mammal, comprising:
orally administering the pharmaceutical composition of claim 1 to the mammal at a dosage effective to reduce the blood glucose in the mammal.

13. The method of claim 1, wherein the pharmaceutical composition is formulated as a tablet or as a preconcentrate.

14. The method of claim 1, wherein the pharmaceutical composition is formulated in a unit dose that provides between 100 and 1,000 mg of the SEDDS or the SEDDS preconcentrate per unit dosage.

15. The method of claim 1, wherein the blood glucose is reduced by at least 10% over a time period of at least 6 hours.

16. The method of claim 1, wherein oral administration reduces HbA1c by at least 0.5% (absolute) when administered over at least 8 weeks.

17. The method of claim 1, wherein administration is once daily.

18. A pharmaceutical composition, comprising:
an incretin or fragment or analog thereof, optionally covalently bound to a carrier protein;
wherein the incretin or fragment or analog thereof is encapsulated in a pheroid drug delivery system.

19. The composition of claim 18, wherein the incretin or fragment or analog thereof is CJC-1134.

20. The composition of claim 19, wherein the CJC-1134 is covalently bound to a carrier protein, wherein the carrier protein is albumin.


SEQUENCE LISTING (1 sequence)

SEQ ID NO: 1 - protein, 40 aa, synthetic construct
HGEGTFTSDLSKQMEEEAVRLFIEWLKNGGPSSGAPPPSK